(19) United States
(12) Patent Application Publication
(10) Pub. No.: US 2024/0125770 A1
(43) Pub. Date: Apr. 18, 2024

(54) CELL ANALYSIS

(71) Applicant: University of Dundee, Dundee (GB)

(72) Inventors: Angus Iain Lamond, Dundee (GB); Jason Swedlow, Dundee (GB); Melpomeni Platani, Dundee (GB)

(21) Appl. No.: 18/243,756

(22) Filed: Sep. 8, 2023

Related U.S. Application Data
(63) Continuation of application No. PCT/EP2022/055937, filed on Mar. 8, 2022.

(30) Foreign Application Priority Data
Mar. 10, 2021 (EP) 21386020.8

(51) Int. Cl. G01N 33/50

(52) U.S. Cl. CPC G01N 33/5073

(57) ABSTRACT

Methods of studying eukaryotic cell responses to a perturbation, or of stratifying eukaryotic cells or cell lines into one or more subgroups are described. The methods involve perturbing a library of cells or cell lines in the same manner, and observing how the cells respond to the same perturbation. The observation may be via a high throughput screening method, for example, cell painting; and the perturbation may be, for example, exposure to a therapeutic agent.

The methods may be used for grouping cells or cell lines that respond similarly to a given therapeutic agent, which may be useful for identifying patient groups and selecting appropriate treatments.

REFERENCE TO RELATED APPLICATIONS

This application is a continuation application of International Patent Application No. PCT/EP2022/055937, filed on Mar. 8, 2022, which claims priority to European Patent Application No. EP 21386020.8, filed on Mar. 10, 2021, the entire contents of each of the above-referenced applications, including all drawings, are incorporated herein by reference.

### FIELD OF THE INVENTION

The present invention relates to methods of studying eukaryotic cell responses to a perturbation, for example, administration of a therapeutic, such as a small molecule drug, or a biological molecule. Aspects of the invention relate to methods involving stratifying cell populations based on variations in such cell responses.

### BACKGROUND TO THE INVENTION

Methods have been developed more than a decade ago that allow the induction of pluripotent stem cells (PSCs), for example from fibroblast cultures derived from both human and mice (1-3). These reports showed that by exogenously expressing a small set of key transcription factors, a terminally differentiated somatic cell could be reprogrammed back into a pluripotent state. This state is characterised by a capacity for self-renewal and ability to differentiate into the three main germ layers, i.e. endoderm, ectoderm and mesoderm. The resulting induced pluripotent stem cells (iPSCs) displayed the key features of their physiological embryonic stem cell (ESC) counterparts, while avoiding most of the ethical issues that have limited the use of stem cells isolated directly from human embryos. Consequently, the use of iPSC lines has attracted great interest as models for human disease and in many applications have replaced the use of hESCs for studies in regenerative medicine and disease modelling, including studies on monogenic disorders and other forms of inherited diseases.

It is known that genetic variations between individuals can result in differing responses to therapeutic treatment—for example, certain cancer drugs are effective against tumours expressing particular cell markers. Where iPSC lines are concerned, gene expression in iPS cells reflects, at least in part, the genetic identity of the donor from whom the cells are derived, and hence may be indicative of responses to therapeutic treatment. It would be beneficial to use this variation in expression and response to obtain a greater understanding of cell and organism responses to therapeutic treatment, and/or to other forms of perturbation.

### SUMMARY OF THE INVENTION

The present disclosure is based in part on observations made by the investigators on work conducted in relation to studying collections of cells of the same cell type obtained from donor organisms. The investigators have observed that although such similar cells may be expected to respond similarly when perturbed in the same way, variations in response can nevertheless be observed. The observation that different cell lines, which are considered to be of the same type, nevertheless display variation in their respective perturbation responses, may permit collections of cell lines to be stratified into sub- groups, displaying similar response profiles. Such observations further allow cells to be used in tests, which reflect, in practice, variation found at the organism level and as may be observed in terms of differential responses to disease susceptibility and/or therapeutic response to a given treatment. For example, the differences in response of human iPSC lines derived from different donors to a cancer drug (for instance, irinotecan, which is used in the clinic to treat colon and small cell lung cancers), may be determined and the different cell lines grouped accordingly.

In one embodiment, there is provided a method of studying eukaryotic cell responses to a perturbation, the method comprising:
providing a library of eukaryotic cells, or cell lines, obtained from a donor, or donors, wherein each eukaryotic cell, or cell line, within the library, is of the same cell type;
perturbing each eukaryotic cell, or cell line, within the library, in the same manner; and
observing how each eukaryotic cell, or cell line, within the library, responds to the same perturbation, in order to identify and group cells, or cell lines, which respond similarly, or differently, to the perturbation.

In a further embodiment there is provided a method of stratifying eukaryotic cells, or cell lines, obtained from a donor, or donors, into one or more subgroups, wherein each of the eukaryotic cells, or the cell lines, are of the same cell type, the method comprising;
perturbing each eukaryotic cell, or cell line, in the same manner and observing how each eukaryotic cell, or cell line, responds to the same perturbation; and
stratifying each eukaryotic cell, or cell line, into said one or more groups, based on the observed response of each eukaryotic cell, or cell line.

In preferred embodiments, the eukaryotic cells are stem cells, such as described further herein; in more preferred embodiments, the stem cells are induced pluripotent stem cells (iPS cells or iPSCs).

A “perturbation” as used herein preferably refers to administering (for example, contacting) a cell or cell line with a test compound. The test compound may be a therapeutic agent or a potential therapeutic agent. For example, the test compound may be a small molecule drug, a biologic, a peptide, protein, a nucleic acid, and so forth. The test compound may in some examples be a potential infectious agent. In some examples, the test compound may be one which is intended to bring about a genetic modification in the cell or cell line. In general, the present specification uses the term “perturbation” to refer to such contacting with test compounds; although it will be appreciated that in certain examples other forms of perturbation may be used; such as exposing the cells to particular environmental conditions or to other cells or cell types.

“Observing a response” or an “observed response” here refers to observing a phenotypic response of the cell or cell line. The phenotypic response may be determined by visual imaging of the cell or of subcellular organelles or components; or may be determined by molecular measurement (for example, identifying changes in gene expression or protein production). This is described further herein.

Preferably, each such group comprises or consists of cells, or cell lines, which respond similarly to the same perturbation.

The following remarks generally apply to each embodiment of the invention, unless otherwise noted.

In accordance with the present invention, the eukaryotic cells, or cell lines, (preferably human in origin, alternatively non-human mammalian cells or cell lines may be used), are of the same cell type. For example, the cells, or cell lines, may be the same type of somatic cell. By way of example, somatic cells, or cell lines, may all be macrophages obtained from blood, kupffer cells obtained from liver tissue, or cardiomyocytes obtained from heart tissue. In preferred embodiments, the cells, or cell lines, may be the same type of stem cell; for example, somatic stem cells, such as hematopoetic stem cells, epithelial stem cells, or mesenchymal stem cells. In more preferred embodiments, the cells, or cell lines, are iPS cells, or cell lines. In some embodiments, the cell, or cell line, is neither diseased, nor phenotypically recognised as being diseased (that is, the cell or cell line does not display a disease phenotype). Thus, in some embodiments, the cells, or cell lines, are generally recognised as being phenotypically disease free. In other embodiments, the cell, or cell line, may carry a genetic feature (such as a mutation, duplication, deletion, of a particular allele), which may predispose a cell, or cell line, to develop a disease or condition. For example, the cells, or cell lines, may be obtained from a donor who, unbeknownst to the donor, and/or unknown to clinical practice at the time of originating the cell line from said donor, may be somehow genetically predisposed to developing a disease, or condition. For example, the cell, or cell lines, may have been taken from a donor, who, at the time of taking the cell, is disease free, but later in life develops a disease/disorder, such as a neurodegenerative, or cardiac disorder. Of course, in some embodiments, the presence of the genetic feature may have been known at the time.

It will be appreciated that when cells are obtained from different donors, although the cells may look (in a phenotypic sense) the same, the cells will possess different genotypes. Even a population of healthy donors, with no known disease-causing mutations, will possess genotypic variation and the present disclosure enables profiling of natural variation in a population, with respect to cell physiology and response to a perturbation. Other forms of variation—for example, epigenetic variations—may also be relevant to different responses to a perturbation and may be investigated using the present methods.

In one embodiment, the cells, or cell lines, may be derived from cells obtained from a donor suffering from a disease, or diseases, associated with either known, or candidate, mutations. Such cells, or cell lines, are profiled and their response to a perturbation compared, with respect to metadata describing the genotype and/or differential disease severity in the donors.

Stem cells may include embryonic, foetal and adult stem cells and may be phenotypically pluripotent, multipotent or unipotent. In one embodiment of the present disclosure, the eukaryotic cells, or cell lines, are, comprise, or consist essentially of, induced pluripotent stem cells (iPSCs). iPSCs are a type of pluripotent stem cell generated directly from a somatic cell. iPSCs are well known and methods for obtaining iPSCs are described, for example in, Kim D, Kim CH, Moon JI, et al. Generation of human induced pluripotent stem cells by direct delivery of reprogramming proteins. Cell Stem Cell. 2009; 4(6):472-476. doi:10.1016/j.stem.2009.05.005, or in references cited therein.

An “iPSC-derived cell,” as used herein, refers to a cell that is generated from an iPSC, either by proliferation of the iPSC to generate more iPSCs, or by differentiation of the iPSC into a different cell type. iPSC-derived cells include cells not differentiated directly from an iPSC, but from an intermediary cell type, e.g., a neural stem cell, or a cardiac progenitor cell. iPSC-derived cells may also encompass cells that have been artificially modified to alter the genotype; for example, by the use of genome editing means, such as either CRISPR-Cas9, or TALENs, to introduce desired modifications to the genome; or by other genetic engineering techniques, which will be known to the skilled person, such as recombinant DNA technologies.

Sources of suitable iPSC lines, or panels of iPSC lines, for use in certain of the methods described herein, may include iPSC lines, or panels of iPSC lines, derived from donors that meet one or more pre-determined criteria. In some cases, donors and cellular samples from such donors, may be selected for the generation of induced stem cell lines and panels of induced stem cell lines, based on one or more of such pre-determined criteria. These criteria include, but are not limited to, either the presence, or absence, of a known health condition in a donor (e.g, spinal muscular atrophy, Parkinson's disease, or amyotrophic lateral sclerosis), one or more positive diagnostic criteria for a health condition, a family medical history indicating a predisposition or recurrence of a health condition, the presence, or absence, of a genotype associated with a health condition, or the presence of at least one polymorphic allele that is not already represented in a panel of induced stem cell lines. In some examples, a panel of cell lines may be used that reflect human genotypes previously linked with susceptibility to certain types of disease.

The investigators have observed that (i) iPSC lines from different human donors, including both healthy donors, or donors with disease causing mutations, vary in terms of protein and mRNA expression patterns; (ii) that lines from the same donor are more similar with respect to levels of protein and RNA expression than lines from different donors; and are able to (iii) detect expression of a large array of proteins within iPSCs that are relevant to disease mechanisms and drug targets in terminally differentiated cell types of patients. It is unexpected that many of these proteins (e.g. proteins encoded by genes relevant to Parkinson's Disease patients), were already expressed and detectable at the protein level in iPSC lines in their pluripotent, undifferentiated state.

The invention involves stratifying cells, or cell lines, based on an observed response to a perturbation. In some embodiments, observing a response may involve comparing an initial cell, or cell line condition, with a post-perturbation condition. For example, image-based phenotypic measurements of the cells, before and after perturbations with drugs, may be used to classify cells into groups, based upon how they respond to the perturbations, as judged by analysis of the imaging data. Further, molecular descriptions of one or more representative lines in each response group may be analysed pre- and/or post-perturbation, to allow a deeper classification of how each group responds to the perturbation at the molecular level, thereby gaining additional insight into the mechanism of response elicited by the drug and how this relates to variation in genetic background.

In one embodiment, pluripotent stem cells (such as iPSCs), derived from healthy male and female donors are compared to identify generic and specific differences in their pre-perturbation condition and their respective responses to perturbations, related to sex or gender, as revealed by imaging and/or molecular descriptions, as described herein. In another embodiment, cells derived from healthy female donors, showing differential levels of X Chromosome Inactivation (XCI), may be obtained and compared to identify differences in their pre-perturbation condition and their respective responses to perturbations, related to XCI levels, as revealed by imaging and/or molecular descriptions, as described herein.

The cells, or cell lines, are subject to the same perturbation in order to identify similarities, or variation of response, to the perturbation. A perturbation may include, either alone, or in combination, contacting the cells, or cell lines, with a small molecule drug compound, or candidate drug compound, or natural product; a protein/peptide, or RNA macromolecule; genetically modifying the cell, or cell line, by, for example, gene product depletions, gene mutations, knockouts and knock-ins; or contacting the cell with an infectious agent, for example a virus, a bacterium and so on.

Observing how each cell responds to the perturbation can be by any suitable method. The “observed response”, may be a visually-observed response, for example, based on imaging of a cell, or cell line. This may be referred to as an image-based phenotypic measurement of the cell. This may involve use of expert systems, or algorithms (with or without manual intervention), to detect and identify subcellular components and observing a response in a specific subcellular component. In other embodiments, an “observed response”, may be a non-visually observed response; for example, gene expression, or “molecular descriptions”, may be analysed. In some embodiments, both visual and non-visual responses may be combined. A “molecular description”, of a cell may include any, or all, of genomic DNA sequences of the lines, expression data for mRNA and/or proteins, potentially including additional molecular descriptions, such as PTMs (post-translational protein modifications), protein complexes ('interactomes), DNA methylation patterns and the like. The observation may be carried out at a single time point, or multiple points over time. The observations may be qualitative and/or quantitative and may involve a plurality of different observations being conducted in order to obtain a phenotypic profile of said cells.

Typically, said analysis may involve in situ cell morphology analysis. This may include an image-based assay, where subcellular components, such as organelles (e.g. cell wall, nucleus, Golgi apparatus, mitochondria, endoplasmic reticulum, iysosomes), macromolecules, membranes, or metabolites, may be specifically labelled in either a cell, or cell line, in the fixed, or living state and detected by a digital imaging system, for example, such as an automated fluorescence imaging system, using, for example, either higher resolution wide-field, confocal, light sheet, or super-resolution fluorescence microscopy, or automated, high-content screening systems. Observations may be carried out in situ (that is, on either living, or fixed cells), without extracting material from the cell. Optionally, or alternatively, the cells may be lysed and extracted material obtained for analysis.

Labelling, if employed, may comprise the use of, for example, either cytochemical staining reagents, or other cellular dyes, one or more labelled antibodies, or other reagents for immunolabeling the cells, or specific cellular components, such as organelles (e.g. cell wall, nucleus, Golgi apparatus, mitochondria, cytoskeleton, endoplasmic reticulum), macromolecules, proteins and the like, a fluorescent reporter protein fusion, or the use of a post-translational modification (PTM)-specific antibody, or any combination of these labelling methods. In certain embodiments, the method may make use of cell painting to label subcellular components; cell painting is described in, for example, reference (8).

Many analysis methods are known in the art and one or more may be used in connection with this disclosure. These include, for example, cytological staining, immunolabelino (immunohistochemistry, immunofluorescence), in situ hybridisation, or other molecular techniques, spectrophotometry, laser microdissection, followed by nucleic acid extraction and analysis (such as by POR and related known techniques, hybridization, blotting etc); or protein extraction (and analysis by blotting, microseouencing, etc.).

Cell morphology analysis may involve staining, such as by using hematoxylin and/or eosin staining, May-Grunwald and/or Giemsa staining. Papanicolaou stain, Feulgen stain and all types of staining, and studying cytomorphological details, and analysis of cellular components. Cell morphology analysis can reveal cellular components, including lipids, polysaccharides, proteins, enzymes and other molecules, as well as changes in cellular structure, shape, size and the like. Immunolabeling may permit labelling of cell specific antigens, transcription factors, mutant proteins, or any protein and/or peptide. This may include the separate detection and quantification of distinct protein isoforms, including isoforms encoded by different mRNAs and isoforms formed by post-translational processing, or enzymatic modification, of a common protein precursor, Detecting such components may include the use of antibodies, or antigen binding fragments thereof. The antibodies may be labelled with chemical agents, which may be detected by enzymatic, colorimetric, or fluorescent means, for example. Alternatively, mass spectrometric analysis may permit the detection (and optionally characterization), of proteins, or other macromolecules, of varying sizes.

Proteins, enzymes, peptides and the like, may be assayed, by, for example, mass spectrometry, to systematically detect and quantify known protein post-translational modifications (PTMs), including, but not limited to, phosphorylation, methylation, acetylation, hydroxylation, glycosylation, or peptidyl modifications, such as ubiquitinylation, SUMOylation, NEDDylation and related peptide modifiers. Additionally, it may be possible, or desirable, to detect, quantify and/or compare protein complexes, using techniques such as, but not limited to, Size Exclusion Chromatography (SEC), ultracentrifugation, or density gradient centrifugation, affinity chromatography, affinity tagging, such as BiolD, or APEX and their variant technologies.

In accordance with the present disclosure, comparison of a plurality of morphological and/or molecular data from panels of cells, such as iPSCs, from different donors, can potentially profile in a laboratory, or pre-clinical trial setting, anticipated differential responses in the human population, pertaining to relevant clinical issues, such as drug efficacy, sensitivity, biomarker variation, or sensitivity to infectious agents.

In one embodiment, the present disclosure enables panels of pluripotent cells, such as iPSCs, derived from male and/or female donors, including healthy donors with no known disease causing mutations, to be compared using molecular screening technologies, such as, but not limited to, high content microscopy imaging, to identify stratified subsets of iPS cell lines, showing common responses of defined molecular markers to perturbation, i.e., either minimal, or no response, or defined response involving a change in cell state (e.g. stress response, cell cycle arrest and/or apoptosis, or other form of cell death).

In a further embodiment, where cells, or cell lines, have been stratified into one or more sub-groups, the stratified cells may be further correlated with genetic metadata, e.g. either specific genetic, or epigenetic markers, including identified combinations of SNPs, levels of XCI, DNA or chromatin methylation, or other recognisable protein, or DNA modifications indicative of epigenetic state.

In a further embodiment, where cells, or cell lines, have been stratified into one or more sub-groups, the stratified cells may be further analysed, using technologies including, but not limited to, mass spectrometry, to identify and characterise protein signatures correlated with defined responses to specific perturbations. The protein signatures may correspond to variation in expression levels of individual proteins, or combinations of proteins, including combinations of specific protein isoforms and/or PTM modified protein variants and/or specific combinations of multi-protein complexes, or other complexes, such as nucleic acid-protein complexes.

The present teaching and methods may find particular application in the design of clinical trials, including pre-selection of patient cohorts and testing of specific therapeutics and formulations. In this regard, cells from potential clinical trial subjects are obtained and subjected to one or more methods, as described herein. Prior knowledge resulting from the analyses, as described, may inform a clinical trial investigator of either a possible, or likely outcome, when the same perturbation is conducted on a subject having a similar profile to the subject from which the cells were obtained. In this manner, the most suitable, or appropriate subjects, may be identified for inclusion in a clinical trial, or outlier subjects identified, so that they can be taken into account when conducting a clinical trial. That is, for example, a potential clinical trial subject may have a cell, or a sample taken from their body, molecularly profiled and compared with molecular profiles from a set of cells, or cell lines, stratified as described herein; and the potential subject either selected, or not selected for the trial, based on an expected stratified group (eg, a group with an expected desired response to a given test compound). In some embodiments the potential clinical trial subject may be the same subject as that from which one or more of the stratified cells, or cell lines, were obtained.

Similar approaches may permit selection of an appropriate therapeutic intervention for a given patient, where the patient's molecular profile can be compared with those from a stratified cell, or cell line, having a desired response either to that intervention, or a similar intervention.

Conducting the methods of the present disclosure will generate data from individual cells and cell lines, as well as panels, or sub-groups of cells/cells lines. It may be possible to perform computational analyses, including deep learning and related artificial intelligence and machine learning strategies, to assimilate and/or link the output data from the analyses, as described herein and use such information when analysing cells from other subjects. For example, the data may be of use in predicting clinical decisions with respect to diagnostics, evaluation of disease progression and/or patient stratification for therapeutic strategy.

### BRIEF DESCRIPTION OF THE FIGURES

FIG. 1 shows the general process of the genetic-informed screening platform. A: Donors, consisting of healthy individuals and patients from the cohorts. B: Skin fibroblasts from different donors are reprogrammed into hiPSCs and are combined into a panel. Each hiPSC line exhibits the unique molecular and cellular properties that reflects the donors' genetic identities. C: hiPSC panels are treated with the drug candidate molecule of interest. D: Cell painting assays are then conducted to assess the phenotypic response of each hiPSC line. E: Consequently, hi PSCs from different donors are clustered based on phenotypes caused by the drugs or drug candidates.

FIG. 2 shows maintenance of pluripotency in hiPSC lines in HCS assay format. hiPSC lines were plated in 384 well plates, fixed and stained with marker antibodies and imaged in a multiwell imager (see Materials and Methods).

FIGS. 3A and 3B show (FIG. 3A) an overview of the cell painting process. Approximately 6000-8000 cells are analysed per donor/drug combination. For every cell, approximately 900 quantitative features are analysed. This includes quantitative features related, but not-limited to, cells, nuclei, organelles, and cytoplasm. Data clustering and reduction are performed on the quantitative data, and the processed data are visualised. (FIG. 3B) images of two cell lines used (shown here are aowh2 and datg2), treated with DMSO, etoposide, and vanquin respectively. Each row shows different cellular components as indicated.

FIGS. 4A-4B show UMAP Embedding of Cell Painting Features. (FIG. 4A) Plot shows the UMAP embedding for features measured for each human iPSC line when treated with the different drugs. Well separated clusters are indicated: (1) DMSO and weak responders; (2) Atorvastatin, Simvastatin, Fluphenazine; (3) Afatinib, Erlotinib, Everolimus and Rapamycin; (4) DNA damaging agents and topoisomerase inhibitors; (5) non-microtubule targeting cytotoxics; (6) Microtubule polymerization effectors. Note the differential response of some lines to Erlotinib. (FIG. 4B) Zoom of cluster 3. Note the splitting of human iPSC lines treated with erlotinib bwteen afatinib and rapamycin and everolimus clusters.

FIGS. 5A and 5B show variations in the magnitude of drug responses across human iPSC lines. (FIG. 5A) Heatmap of deviations from DMSO for each human iPSC line. Example of one possible visualisation. The heatmap shows the number of deviations for each feature from DMSO for each human iPSC line. The response to each drug creates a characteristic pattern of features, but with differences in the magnitude of response. Responses to differential responses by each cell features among different hiPSC lines when treated with the same drug (in this case, Atorvastatin (top) and Rapamycin (bottom) are shown). (FIG. 5B) Induction plot of drug responses. Variations are plotted as the fraction of features for each combination of drug and human iPSC line that are >3 σ (standard deviation) away from the DMSO (control). DMSO is highlighted in the green box.

FIGS. 6A-6C show (FIG. 6A) volcano plot of proteins showing differential expression in the human IPSO lines stratified by atorvastatin response. Representative proteins involved in lipid metabolism are shown in red. B. and C. GO Term Enrichment Analysis showing volcano plot (FIG. 6B) and bar chart (FIG. 6C) of gene sets and pathways showing differential expression in the human IPSO lines stratified by atorvastatin response. Note pathways related to lipid metabolism. GO analysis p-values of the above pathways/gene sets are included in Table 4.

FIG. 7 shows ErbB Pathway Responses. A. UMAP and nuclear image of 4 different hiPSC lines (zaie1, paim3, pelm3, and voce2) treated with Erlotinib. Differential response can be observed, where zaie1 are in the weak responders group, paim3 and pelm3 in the afatinib cluster, and voce2 (along with the majority of hiPSCs treated with Erlotinib) in the Erlotinib cluster on the bottom right of the UMAP. B. EGFR Inhibitor Pathway diagram. EGFR signalling pathway plays a major role in regulating cell division and survival. EGF receptors are large transmembrane glycoproteins consisting of an N-terminal ligand binding domain, a hydrophobic transmembrane domain and an intracellular cytoplasmic tyrosine kinase domain. Receptor activation occurs by binding to different ligands. Dimerisation of the receptor induces the activation of its kinase domain and subsequent activation of a cascade of signalling pathways including the Ras/ERK , JAK/STAT and PI3K/Akt pathways. Erlotininb is a specific tyrosine kinase inhibitor for EGFR, while Afatinib inhibits all HER family receptors. Rapamycin and Everolimus are mTOR pathway inhibitors. Note the clustering of the cell lines in (A) based on similar pathway inhibition.

### DETAILED DESCRIPTION OF THE INVENTION

Reference panels of human iPSC lines have been established, which provide a resource for analyses in pluripotent cells. In particular, the Human Induced Pluripotent Stem Cell Initiative (www.hipsci.org), have established a library of human iPSC lines, including many lines derived from healthy donors, as well as from patient cohorts with known genetic disorders (4). The HipSci cell lines were derived from donors of varied age and both male and female gender and were all reprogrammed from human skin fibroblasts, following transduction of the pluripotency transcription factors using a Sendai virus vector (4). Many of these cell lines have been subjected to poly-omic and phenotypic analysis. Importantly, data from both mRNA and protein level analysis of gene expression in these lines show that, at least in part, gene expression in the iPS cells reflects the genetic identity of the human donor. Consequently, two or more iPS lines derived from the same individual are typically more similar to each other in their gene expression profile, than they are to lines derived from different donors (4, 5). In addition, analysis of the iPSC proteome has recently shown that induced stem cell lines express proteins from a very broad range of genes (5, 6). This represents a larger fraction of total human protein coding genes than are typically detected being expressed in either terminally differentiated, primary cells and tissues, or in most tumour-derived transformed cell lines. In total, iPSC lines show expression of >16,000 protein groups that can be detected by MS analysis, with a median protein sequence coverage of ˜46% across all proteins, with this depth of protein coverage relatively constant across chromosomes. For the majority of chromosomes, >50% of their known protein coding genes are expressed in iPSCs, spanning a wide dynamic range of expression levels from <100 to ˜100 million copies per cell. Further, the human iPSC proteome shows comprehensive coverage of known protein complexes, including subunits from ˜92% of all complexes described in the mammalian protein complex database, CORUM10, as well as most protein families involved in cell signalling. This includes ˜74% of all confirmed human kinases, ˜70% of known protein phosphatases and ˜66% of known E3 ubiquitin ligases. Notably, with respect to many cell signalling pathways targeted by anti-cancer drugs, iPSC lines express ˜2.5-fold more receptor tyrosine kinases than many primary T cells or other tumour cell lines. As a result of this high level of protein expression, undifferentiated iPSCs are thus better suited to screening compound libraries with a broad range of targets than the cancer cell lines currently used for phenotypic screening by Pharma.

In summary, recent studies on the gene expression and phenotypic behaviour of multiple iPSC lines derived from many distinct healthy human donors, indicate that these cells, even in the undifferentiated, pluripotent state, exhibit properties at the molecular and cellular level reflecting the varied genetic identities of the original donors. This raises the possibility that through screening the responses of panels of cells derived from different human donors, it may be possible to identify examples where drug-cell interactions and phenotypic outcomes, can be predicted in the laboratory and used to inform and thereby improve, the outcomes of downstream clinical trials and/or therapeutic or diagnostic procedures, or decisions, used in the clinic.

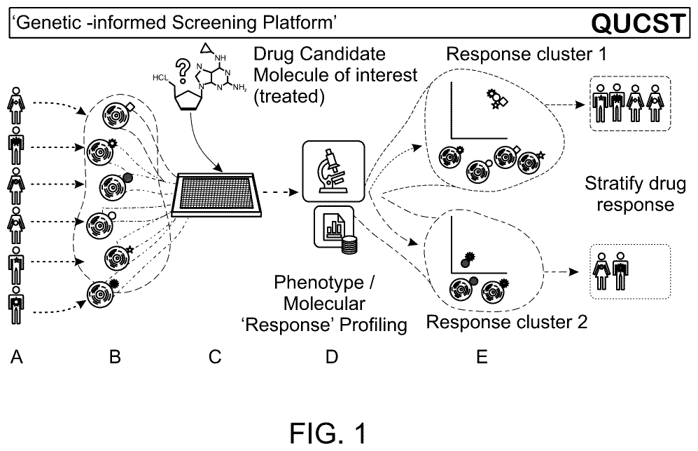

As described below, one promising recent technology approach for the cost-effective, high-throughput, quantitative screening of cell responses, involves microscopy-based fluorescence imaging, also known as ‘cell painting’ (7, 8). We have therefore used an implementation of the cell painting technology to characterise whether panels of human iPSC lines, derived from different donors, show variation in their phenotypic responses to a range of small molecule inhibitors and known drugs, including therapeutics in clinical use (FIG. 1). Specifically, we have explored how this can be used to create a ‘genetics-informed screening platform’, allowing a data-driven stratification of a library of genetically distinct human cell lines, based upon information extracted from the analysis of multi-channel fluorescence microscopy images, following the treatment of the panels of cell lines with different drugs. Furthermore, we explore how this strategy of image-based, phenotypic response stratification can be combined with analysis of the genotypes of the respective stratified cell lines and their detailed mRNA and protein expression profiles, to determine potential molecular mechanisms underlying the observed differences in drug-cell interactions.

### MATERIALS & METHODS

### Cell Culture

Human iPSC lines used in this study were from the HipSci cohort as previously described (4). Feeder-free human iPSC lines were cultured in Essential 8 (E8) medium (E8 complete medium supplemented with (50×) E8 supplement ThermoFisher-A1517001) on tissue-culture dishes coated with 10 μg/cm² of reduced Growth Factor Basement Membrane matrix (Geltrex, ThermoFisher A1413202 resuspended in basal medium DMEM/F12 ThermoFisher 21331020). Medium was changed daily.

To passage feeder-free hiPSC lines, cells were washed with PBS and incubated briefly 3-5 min with 0.5 mM PBS-EDTA solution. The PBS-EDTA solution was removed, cell clusters were resuspended in E8 medium and seeded at ratios of 1:4 to 1:6 depending on their confluency on Geltrex-coated tissue culture dishes. Established and undifferentiated feeder-free human iPSC lines were expanded and frozen prior to transition to TeSR medium for further study. To transition to TeSR medium supplemented with bFGF (Peprotech 100-1813, at a final concentration of 30 ng/m1), Noggin (Peprotech 120-10C, at a final concentration of 10 ng/m1), Activin A (Peprotech 120-14P, at a final concentration of 10 ng/ml), E8 medium of hiPSC lines was exchanged daily to medium containing increasing amounts of TeSR medium until human iPSC lines were completely maintained in TeSR medium alone.

To passage human iPSC lines transitioned to TeSR medium, cells were washed with PBS and incubated briefly with TrypLE Select (ThermoFisher 12563029) to create a single cell suspension prior to resuspension in TeSR medium supplemented with ROCK kinase inhibitor (Tocris 1254, at a final concentration of 10 μM), bFGF (Peprotech 100-1813, at a final concentration of 30 ng/ml), Noggin (Peprotech 120-10C, at a final concentration of 10 ng/ml), Activin A (Peprotech 120-14P, at a final concentration of 10 ng/ml) and seeded at a concentration of 5×10⁴ cells/ml on Geltrex-coated tissue culture dishes. Pluripotency of TeSR transitioned hiPSC lines was verified by immunofluorescence prior to cell banking or experimental use. Medium was changed daily.

ID number and details of the cell lines are listed in Table 1.

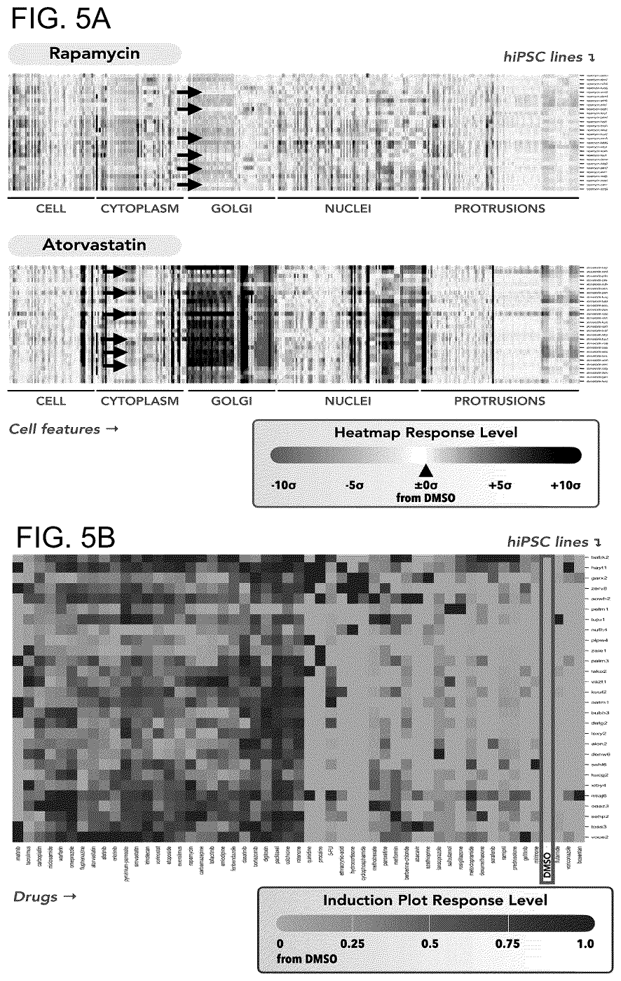

TABLE 1
List of HipSci lines used in this study
Symbol represent all figures in this document except FIG. 5A

| Cell ID | Symbol | Donor Description |
|---|---|---|
| aion2 | [symbol] | White British, Male |
| aowh2 | [symbol] | White British, Female |
| babk2 | [symbol] | White British, Female |
| bubh3 | [symbol] | White British, Female |
| datg2 | [symbol] | White British, Female |
| denw6 | [symbol] | White British, Male |
| garx2 | [symbol] | White British, Female |
| hayt1 | [symbol] | White Other, Male |
| kucg2 | [symbol] | White British, Male |
| kuul2 | [symbol] | White British, Male |
| lako2 | [symbol] | White British, Female |
| lexy2 | [symbol] | White British, Female |
| miaj6 | [symbol] | White British, Male |
| nufh4 | [symbol] | White British, Female |
| oaaz3 | [symbol] | White British, Male |
| oatm1 | [symbol] | White British, Male |
| paim3 | [symbol] | White British, Male |
| pelm1 | [symbol] | White British, Female |
| pipw4 | [symbol] | White British, Male |
| sehl6 | [symbol] | White British, Female |
| sehp2 | [symbol] | White Other, Female |
| toss3 | [symbol] | White British, Male |
| tuju1 | [symbol] | White British, Female |
| vazt1 | [symbol] | White British, Male |
| voce2 | [symbol] | White British, Male |
| xiny4 | [symbol] | White British, Female |
| zaie1 | [symbol] | White British, Female |
| zerv8 | [symbol] | White British, Female |

### Cell Staining

384-well tissue culture plates (CellCarrier-384 Ultra Microplates, Perkin Elmer 6057300) were coated with human plasma fibronectin (Merck Millipore FC010) at a concentration of 5 μg/cm². Cells were passaged as described above with TrypLE Select, counted and seeded on the fibronectin coated wells at a concentration of 3×10⁴/cm². Cell line plating was in rows, with three wells per condition for each cell line. Cells were incubated for 24 hrs prior to drug treatment, followed by a further 24 hrs incubation before final fixation and staining and high content imaging. Antibody staining of a separate 384-well plate at 48 hrs confirmed that pluripotency was maintained at 48 hrs.

All fixation, permeabilisation and immunostaining were performed at room temperature. Paraformaldehyde 8% in PBS (PFA, Sigma F8775) was added to an equal volume of medium for a final concentration of 4% and incubated for 15 min. Fixed cells were washed in PBS and permeabilised with 0.1% v/v Triton-X100 (Sigma) for 15 min. Following permeabilisation, cells were washed in TBS and stained with DAPI (Thermo Fisher D1306), SYTO 14 (Thermo Fisher S7576), Concanavalin A (Thermo Fisher C11252), Phalloidin Alexa (Thermo Fisher A12381), and WGA (Thermo Fisher W11262) for 1 hr. Mitotracker (Thermo Fisher M22426) staining was performed in live cells with a 30 min incubation at 37° C. prior to fixation. Cell fixation, permeabilisation, staining and washing steps were performed using automated liquid handling systems (405 plate washer, BioTek—Tempest, Formulatrix).

### Drug Treatment

Drug dosing on 384-well plates was performed using an Echo acoustic liquid handler and corresponding software (Labcyte). Screened compounds were selected from the appropriate chemical library plates containing Cloud library (Enamine) and NPSC Control library. For etoposide drug dosing was performed at 1.7 μM. For all other compounds used in this study drug dosing was performed at 5 μM. Table 2 shows compounds and concentrations used in this study.

TABLE 2
List of drugs used as a candidate
molecule to treat the hiPSC panels

| Drug | Disease target | Concentration | |
|---|---|---|---|
| DMSO (control) | Control | 5 | μM |
| 5-FU | Cancer drug | 5 | μM |
| Abacavir | HIV reverse transcriptase | 5 | μM |
| | inhibitor | | |
| Afatinib | Cancer drug (breast) | 5 | μM |
| Amlodipine | Heart disease | 5 | μM |
| Atorvastatin | Heart disease (coronary) | 5 | μM |
| Azathioprine | Immunosuppressant | 5 | μM |
| Berberine Chloride | Transcription/mitochondrial | 2.68 | μM |
| | function | | |
| Bortezomib (Cloud) | Cancer drug | 5 | μM |
| Bosentan | Heart disease (Pulmonary | 5 | μM |
| | hypertension) | | |
| Carbamazepine | Epilepsy | 5 | μM |
| Carboplatin | Cancer drug (neoplasm/ | 5 | μM |
| | carcinoma) | | |
| Cyclophosphamide | Cancer drug (breast | 5 | μM |
| Monohydrate | neoplasm) | | |
| Dasatinib | Cancer drug | 5 | μM |
| Dexamethasone | Lung condition | 5 | μM |
| Digitoxin | Cancer drug | 5 | μM |
| Erlotinib | Cancer drug (breast) | 5 | μM |
| Ethacrynic Acid | Cancer drug, ALS, | 5 | μM |
| | Parkinson's | | |
| Etoposide | Cancer drug (topoisomerase | 1.7 | μM |
| | inhibitor) | | |
| Everolimus | Cancer drug (breast) | 5 | μM |
| Fenbendazole | Parasitic infections | 3.34 | μM |
| Fluphenazine | Antipsychotic | 5 | μM |
| Flutamide | Cancer drug (prostate) | 5 | μM |
| Gefitinib | Cancer drug (breast) | 5 | μM |
| Hydrocortisone | Steroid | 5 | μM |
| Imatinib | Cancer drug | 5 | μM |
| Irinotecan | Cancer drug | 5 | μM |
| | (topoisomerase inhibitor) | | |
| Lansoprazole | Stomach ulcer | 5 | μM |
| Metformin | Diabetes | 5 | μM |
| Methotrexate | Cancer drug (lymphoma) | 5 | μM |
| Metoclopramide | Nausea/vomiting | 5 | μM |
| Milrinone | Heart disease (heart failure) | 5 | μM |
| Niclosamide | Cancer/bacterial/viral infections | 5 | μM |
| Omeprazole | Stomach ulcer | 5 | μM |
| Paclitaxel | Cancer drug (breast) | 5 | μM |
| Paroxetine | Depressive disorder | 5 | μM |
| Prednisolone | Carotid stenosis | 5 | μM |
| Procaine | Anaesthesia | 5 | μM |
| Pyrvinium Pamoate | Antihelminthic drug with | 5 | μM |
| | anticancer potential | | |
| Ramipril | Heart disease | 5 | μM |
| Rapamycin | mTOR inhibitor | 5 | μM |
| Rosiglitazone | Diabetes | 5 | μM |
| Rotenone | Pesticide | 5 | μM |
| Salbutamol | Bronchodilator | 5 | μM |
| Simvastatin | Coronary disease | 5 | μM |
| Sorafenib | Cancer drug (breast) | 5 | μM |
| Tacrolimus | Immunosuppressant | 5 | μM |
| Tofacitinib | Arthritis | 5 | μM |
| Voriconazole | Anti-fungal | 5 | μM |
| Vorinostat | HDAC inhibitor | 5 | μM |
| Warfarin | Heart disease | 5 | μM |

### Cell Staining of Pluripotency Markers

All fixation, permeabilisation and immunostaining were performed at room temperature. Paraformaldehyde 8% in PBS (PFA, Sigma F8775) was added to an equal volume of medium and cells were incubated for 15 min. Fixed cells were washed in PBS and permeabilised with 0.5% v/v Triton-X100 (Sigma) for 10 min (apart from TRA1-81 staining). Cells were blocked in 10% normal donkey serum for 1 hr prior to antibody incubations. Pluripotency marker antibodies used in this study are listed as follows: Nanog, Oct4, Sox2, Tra-1-81 all at 1:500 (NEB StemLight Pluripotency Antibody Kit 9656). All affinity purified donkey secondary antibodies (either Alexa 488 or Alexa 594) were purchased from Jackson immunoresearch). Hoechst 33342 (Thermo Fisher) was used to stain DNA.

### Imaging

All imaging data were collected on an InCell 2200 high content imager, with a 20× lens and a Quad2 multichroic mirror (GE, Issaquah, WA).

### Data Processing, Analysis and Visualization of Multiple Cell Lines

Raw images were imported into OMERO Plus (Glencoe Software, Inc., (9)) and then processed using a custom pipeline in CellProfiler (8) which segmented nuclei, cytoplasm, golgi and cortical protrusions and calculated a range of defined features. All further steps were executed using the Pandas Python library (10). Features for each plate were normalised by the median of features in DMSO control for that plate. Features whose coefficient of variation was greater than 0.50 were removed from further analysis (typically these were Zernike Phase features). We further removed all features with |Spearman coefficient|>0.98. Z-normalised data were then visualised using principal component analysis (PCA) and Uniform Manifold Approximation and Projection (UMAP (11)). For PCA, we calculated the first 15 principal components and variance included in the first two principal components was 77.1%. UMAP parameters were n_neighbours=15, mindist=0.01 and a cosine metric for scaling distance between points. GO enrichment analysis was carried out using WebGestalt (WEB-based Gene SeT AnaLysis Toolkit) (12)

### RESULTS

### Pluripotency in HipSci Cell Lines in High Throughput Formats

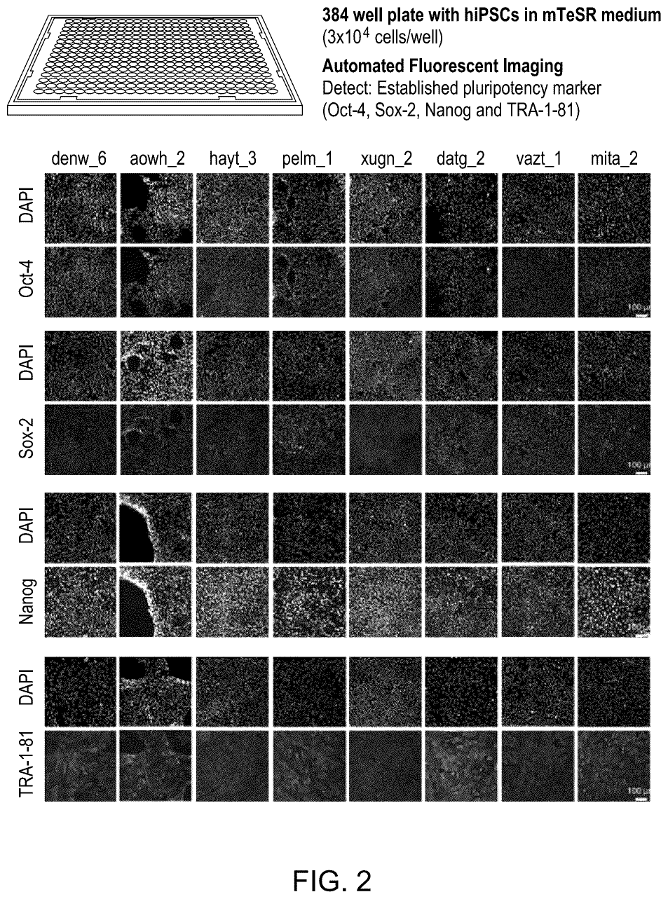

To establish the validity of our assay format using a library of pluripotent iPSC lines from different donors, we determined the stability of different human iPSC lines when cultured in 384 multi-well plates, suitable for use in automated fluorescence imaging. We propagated human iPSC lines in mTeSR medium, plated the cells in 384 multi-well plates (3×10⁴ cells/well), and analysed the cells by immunofluorescence, staining for the established markers of pluripotency, Oct-4, Sox-2, Nanog and TRA-1-81. FIG. 2 shows a representative example of the staining patterns seen for Oct-4, Sox-2, Nanog and TRA-1-81 in eight of the 30 lines analysed in this experiment. All four markers showed strong, relatively homogeneous staining with little or no detectable loss of signal for up to 72 hours after transfer and growth of the cells on the 384 multi-well plates. Strong expression of these pluripotency markers was maintained when cells were plated at a range of concentrations from 25×10⁴ to 75×10⁴ cells/well.

We conclude that human iPSCs can be maintained in multiwell formats suitable for large-scale compound profiling and that the panels of human iPSCs can be maintained in the pluripotent state for the 48-72 hours required for conducting the assay protocol.

### Segmentation and Processing of Multiple Cell Lines

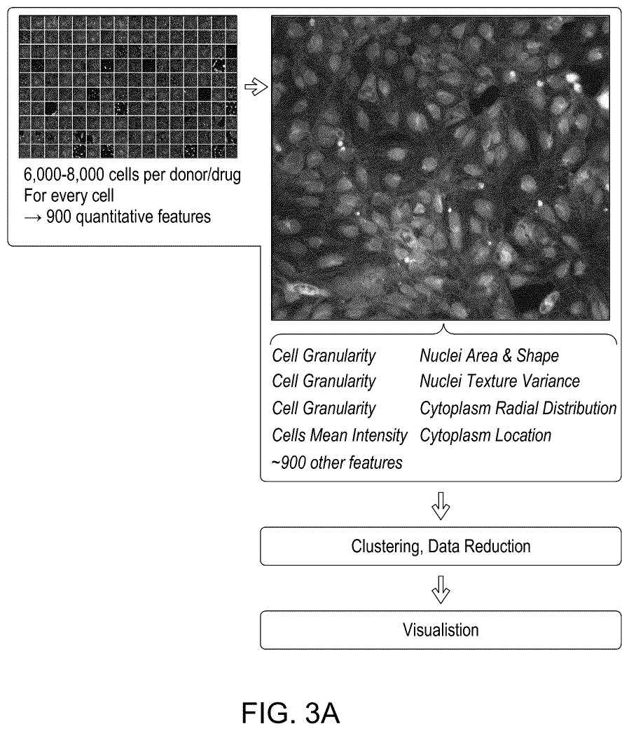

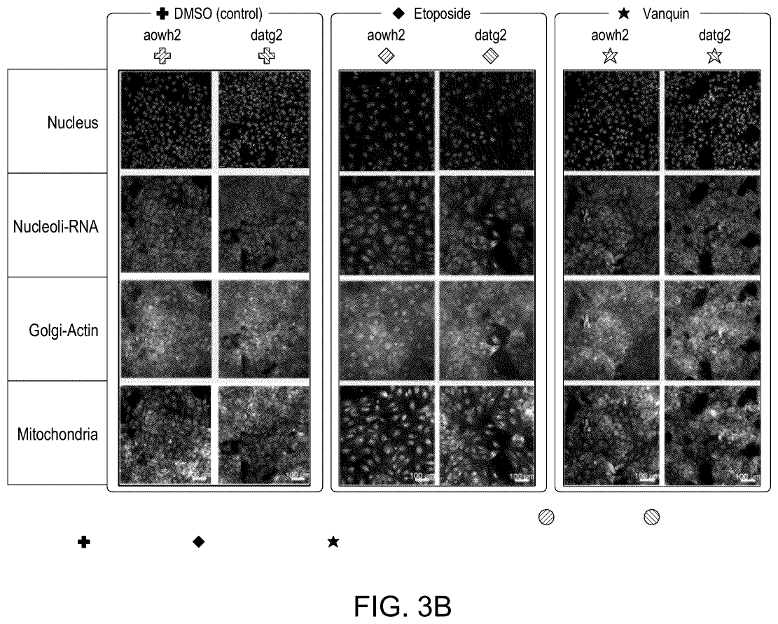

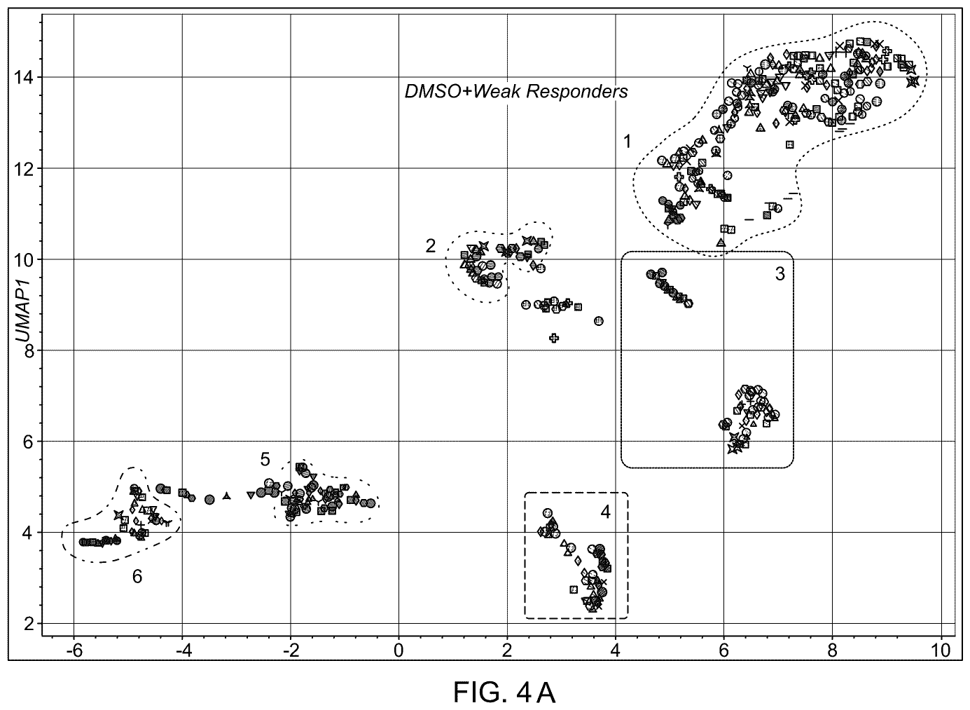

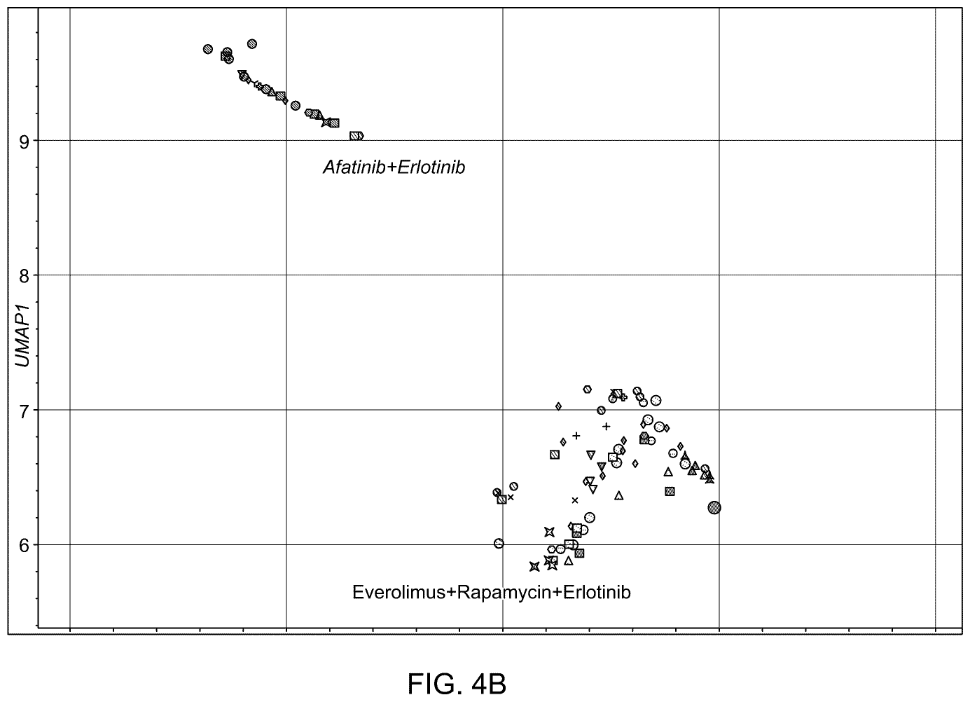

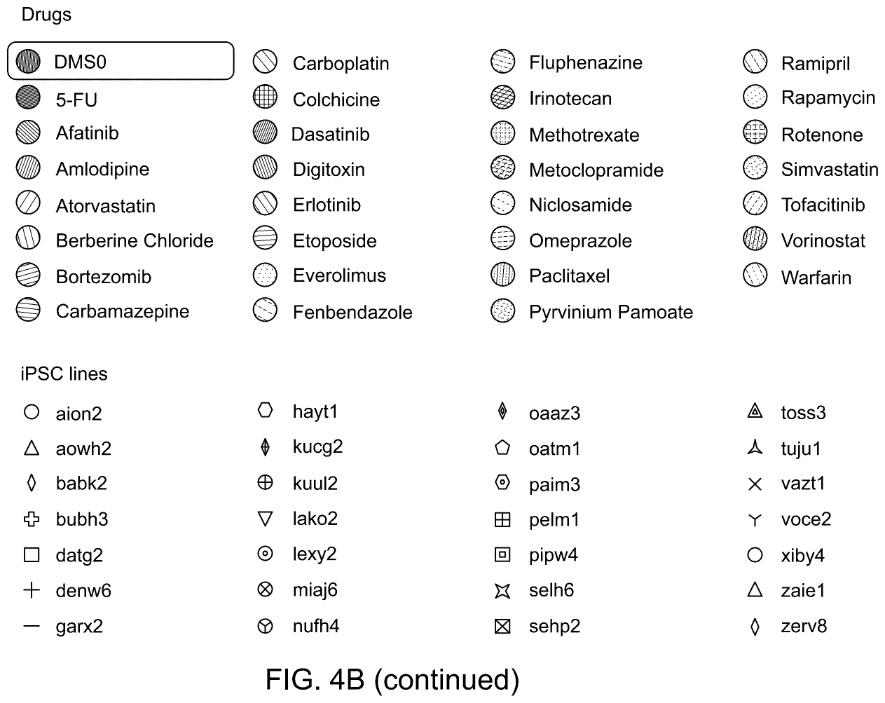

We next used a selection of human iPSCs from the HipSci cohort to test our ability to detect differential effects of FDA-approved drugs, using an image-based cell phenotyping assay. All selected human iPSC lines were previously subjected to whole genome sequencing (https://www.hipsci.org) and the genome data are publicly available. Cells from selected human iPSC lines (Table 1), were grown in 384 well plates, treated with selected compounds (drugs and concentrations used are shown in Table 2), for 24 hrs, then fixed and stained with cell painting markers (see Materials and Methods). Images were recorded in an automated, plate-based imager, then imported into OMERO (9). Images were segmented with respect to the features, ‘nuclei’, ‘cytoplasm’, ‘Golgi’ and ‘protrusions’, using a custom CellProfiler pipeline (13) and image features were then calculated (˜300 features per cell compartment). FIG. 3A shows image thumbnails of a typical experimental plate from a representative experimental plate and an example of the segmentation of individual cells in a single image. FIG. 3B shows enlarged image thumbnails of two cell lines treated with DMSO, Etoposide and Vanquin respectively. To visualise differences in drug responses between human iPSC lines we subjected all measured features to Robust Z normalisation (see Methods) and then expressed all measured features as the number of standard deviations away from DMSO for each human iPSC line. After Z normalisation, we subjected all features and drug/cell line combinations to embedding with UMAP (FIG. 4A). Several compounds cluster according to their known mechanism of action, including simvastatin and atorvastatin (cluster 2), everolimus and rapamycin (cluster 3), topoisomerase inhibitors (cluster 4), cytotoxics (box 5) and microtubule polymerization effectors (box 6). Fluphenazine clusters near the statins (cluster 2), consistent with suggestions that this drug has effects on lipid metabolism of patients prescribed this drug (14) (box 2). DMSO and compounds that cause relatively weak responses at the concentrations used in this assay are shown in cluster 1.

This plot provides several examples where single compounds show varying responses in different human iPSC lines. Most cells responded to 5-FU (cluster 1, red, FIG. 3A), but several showed a response indistinguishable from DMSO, at the concentration tested. We also observed examples of splitting of clusters in human iPSCs treated with vorinostat, and fenbendazole (FIG. 4A). The responses of two cell lines (paim3 and pelm1) to erlotinib, a first generation HER2 inhibitor, are similar to the more specific, second generation HER2 inhibitor afatinib, whereas the erlotinib response in all other human iPSC lines resembles rapamycin and everolimus (see below).

These results suggest that individual drugs may evoke distinct and distinguishable responses in human iPSCs generated from different donors. The differential responses, at least at the level of UMAP embedding, suggest the drugs cause substantially different effects on intracellular pathways and response systems, potentially as a result of normal population level genetic variation between individuals.

Our previous work has demonstrated that variations in protein expression can be controlled by specific genomic loci which are called pQTLs (5). We hypothesised that another potential source of differential drug responses between human iPSC lines might be due to variations in stoichiometry of components of drug response pathways (15). In this case, patterns of drug response features might look similar, but the magnitude of response might differ. To detect differences in the magnitude of responses, we plotted the deviations for all features (before Z normalization) from DMSO, for all cell lines, as a heatmap. FIG. 5A shows heatmaps for atorvastatin and rapamycin, two of the drugs we assayed that showed tight clustering in UMAP embedding (FIGS. 4A-4B). While the patterns of features responses are similar for all human iPSC lines treated with either drug, the magnitude of the responses differ between the different cell lines we assayed.

To visualise differences in response magnitude for a the whole set of assayed drugs and human IPSO lines, we determined, for each pair of drugs and human iPSC lines, the fraction of all features that are >3σ apart from the DMSO control (thus, distinguishable from DMSO with >99% certainty). The resulting plot measures the magnitude of drug response, (referred to as an “induction plot”, FIG. 5B). This plot shows that the same drug and dose gives surprisingly different levels of induction, or response magnitude, across the range of drugs in our test set and that variations in response magnitude are surprisingly common. We also note that these differences are detected despite strong clustering in a nonlinear embedding method, i.e. UMAP, for several drugs, including atorvastatin and simvastatin. We conclude that variable responses are a common feature of human iPSC lines to a wide variety of drugs.

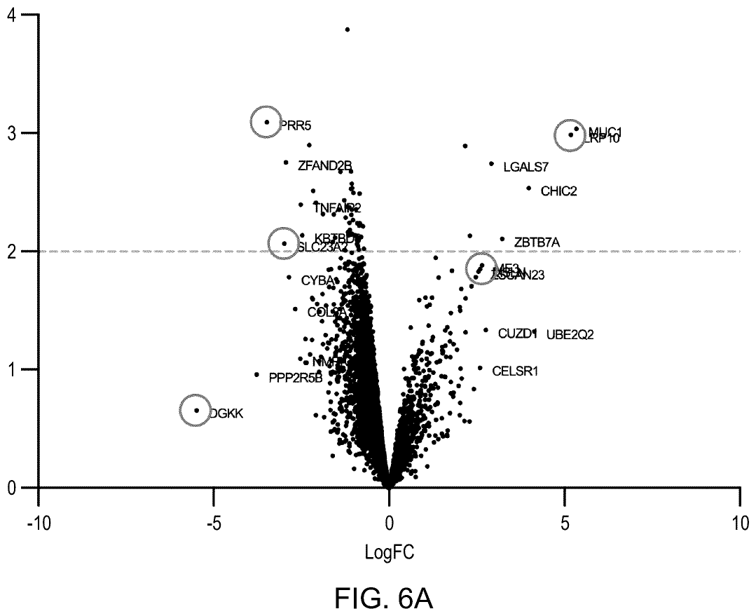

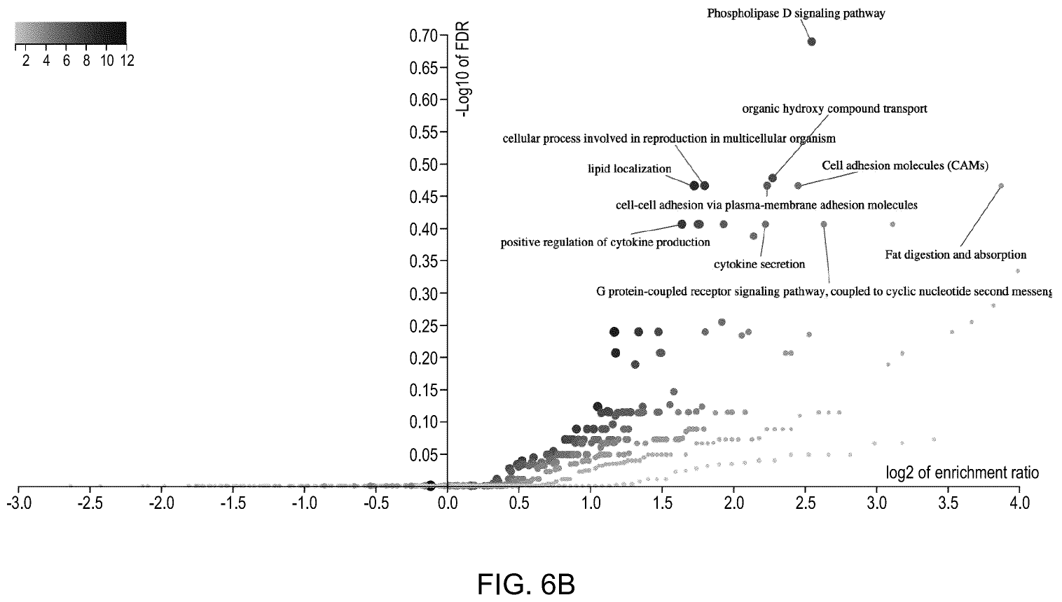

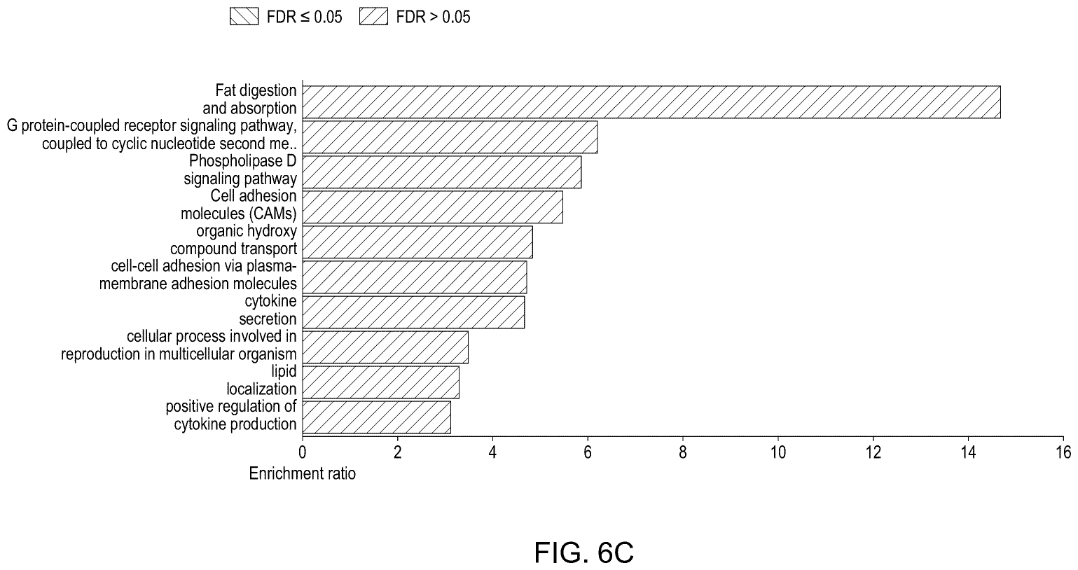

As an initial test of this hypothesis, we used the induction plot to select “low” and “high” responders for a well-characterised drug, i.e., atorvastatin, a HMG-CoA reductase inhibitor. Using previously existing, total proteome data for the chosen human iPSC lines analysed without drug treatment, we calculated log FC vs−log P (so called “volcano plots”) to detect intrinsic differences in protein expression levels between the lines (FIG. 6A).

FIG. 6A shows the differences in protein expression between human iPSC that were stratified based upon measured differences in their response to atorvastatin. Many of the proteins that show a differential expression level are involved in lipid metabolism. We confirmed this result using GO term enrichment analysis and observed a significant enrichment for expression of protein factors involved in lipid metabolism when comparing the cell lines stratified by response to atorvastatin (FIGS. 6B and 6C). These data are consistent with the variation in response to atorvastatin between iPSC lines resulting from, at least in part, differences in the expression levels of one or more proteins involved in lipid metabolism.

TABLE 3
Proteins involved in lipid metabolism showing differential
expression in the human IPSC lines stratified by atorvastatin
response (FIG. 6A- volcano plot, red circles).

| Protein | Function |
|---|---|
| DGKK | Master in lipid metabolism controlling ratio of |
| | DAG/PA levels |
| PRR5 | Member of mTORC2 complex, role in metabolic |
| | reprogramming |
| SLC23A2 | Vitamin C transporter (vitamin C stimulates |
| | cholesterol synthesis) |
| MUC1 | Glycoprotein, role in uptake and absorption of |
| | cholesterol |
| ORP2 | Oxysterol -byproduct of cholesterol binding |
| | protein |
| Mitochondrial | Several roles including role in lipid accumulation |
| malic enzyme 3 | in mammals |
| LRP10 | Cholesterol uptake receptor |

TABLE 4
GO Term Enrichment Analysis of gene sets/pathways
and their associated p-values from FIGS. 6B and 6C.

| Gene Set - Description | P-Value |
|---|---|
| Phospholipase D signalling pathway | 0.00017761 |
| Organic hydroxy compound transport | 0.00057855 |
| Fat digestion and absorption | 0.00096508 |
| Lipid localisation | 0.0015540 |
| Cell-cell adhesion via plasma-membrane | 0.0016540 |
| adhesion molecules | |
| Cellular process involved in reproduction of | 0.0020457 |
| multicellular organism | |
| Cell adhesion molecules CAMs | 0.0020806 |
| G protein-coupled receptor signalling pathway, | 0.0037523 |
| coupled to cyclic nucleotide second messenger | |
| Positive regulation of cytokine production | 0.0040415 |
| Cytokine secretion | 0.0041795 |

Linking Variable Responses to Intracellular Pathways The EGFR signalling pathway plays a major role in regulating growth, cell division, differentiation and cell survival. The pathway is controlled by the activity of the HER family of transmembrane receptors (comprising four members, i.e., EGFR, HER2, HER3 & HER4), which are known to be activated by binding of different extracellular ligands. Ligand binding stimulates receptor dimerisation and induces the activation of a cytoplasmic tyrosine kinase domain. This leads to the activation of a cascade of signalling pathways including the Ras/ERK, JAK/STAT and P13K/Akt pathways. Given the involvement of EGFR in so many diverse cellular processes, and the fact that aberrant activity of EGFR has been shown to play a major role in the development and growth of tumour cells, several drugs have been developed to interfere with EGFR activity (16, 17).

Two examples of approved drugs that target EGFR family members are Erlotinib and Afatinib, synthetic molecules that block ligand-induced receptor autophosphorylation by binding to the ATP binding pocket in the cytoplasmic tyrosine kinase domain of the receptor.

Erlotinib is reversible and specific for EGFR, while Afatinib is an irreversible inhibitor and inhibits all homo- and heterodimers formed by EGFR, HER2, HER3 and HER4 family receptors. Afatinib not only irreversibly inhibits signal transduction of all members of the EGFR family, but also inhibits the phosphorylation of downstream signal transduction molecules, such as ERK and Akt (18).

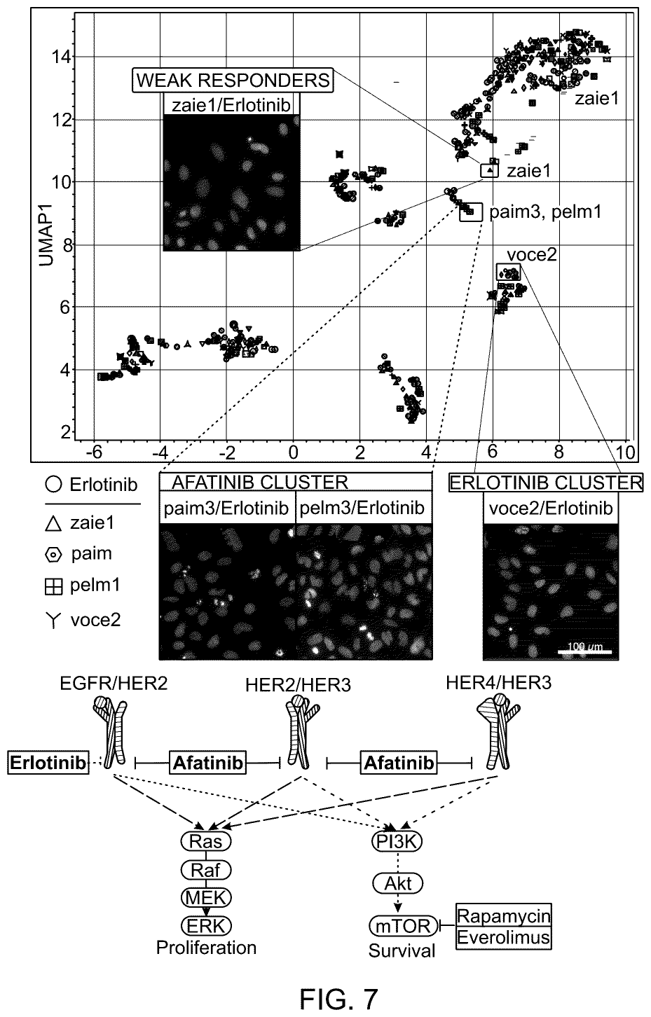

In our cell painting assays, we observed that responses to erlotinib and afatinib were, in most cases, clearly distinguished in a UMAP plot (FIG. 4A box3, FIG. 4B and FIG. 7). We observed that all human iPSC lines (except for one, zaie1, see FIG. 7) treated with afatinib formed a single cluster distinct from all other tested drugs, suggesting a uniform response across 27 human iPSC lines. By contrast the response of the majority of human iPSC lines treated with erlotinib clustered with rapamycin and everolimus, suggesting that in these lines, the main effect of erlotinib is to inhibit intracellular pathways that activate mTOR signalling. Two erlotinib-treated cell lines, paim3 and pelm1 clustered with afatinib suggesting a broader effect on intracellular pathways, similar to afatinib. These data suggest a hypothesis that the difference in response in paim3 and pelm1 cell lines are due to EGFR receptor mutations in the germline, partial ErbB pathway silencing, or most likely, or different levels of inhibition of ERK and/or Akt (see FIG. 7). Further studies using quantitative proteomics can be used to provide a mechanistic insight into these results.

### DISCUSSION

The Nobel-winning discovery of a mechanism allowing terminally differentiated human cells to be re-programmed back to a pluripotent state that resembles embryonic stem cells (ESCs), has opened exciting new possibilities to study human disease and drug responses, while overcoming many of the ethical issues associated with using ESCs (2). The creation of such human induced pluripotent stem cells (hiPSCs), which can be generated from a wide range of different healthy donors and/or disease cohorts, now opens the door to new, laboratory-based methods for analysing drug-cell interactions and how this varies as a result of genetic variations within the human population.

We have developed the first such ‘genetically-informed’, high-throughput assay for assessing variable drug responses in human cells. This leverages the recent discovery that gene expression analyses, at both the RNA and protein levels, show that these molecular markers in human iPSC lines derived from different donors reflect, at least in part, the genetic identity of the donor (5). Thus, by screening how panels of iPSC lines from different donors respond to drug treatment, in a standardised assay format, we can stratify the lines based upon objective measurements of their varied phenotypic responses. Using the image data from a ‘cell painting’ fluorescence microscopy assay, features can be extracted and used to stratify the different cell lines, reflecting the differences in their response to the drug treatments, as illustrated in FIG. 2. Further in-depth molecular analyses, particularly at the proteome level, can then be performed to compare the different stratified groups of cell lines and help understand the mechanistic basis for the differential phenotypic responses observed. Proteome level analysis is expected to be most informative here, because proteins are the targets of most drugs in clinical use. Proteins are also the mediators of most disease mechanisms and the mechanisms of drug action.

Variable Responses of hiPSCs to FDA Approved Drugs: As summarised above, we have created and validated an innovative drug screening technology platform that uses panels of human iPSC lines established from different donors to sample genetic diversity in the human population. The cell lines are analysed in high-throughput, cell painting fluorescence microscopy assays. This cell painting assay can identify and quantify how clinically relevant drugs alter the phenotypes of cells and allow comparisons to be made in how different iPSC lines respond to the same drug treatment.

When treated with the vehicle (DMSO) control, each of the cell lines show a tightly clustered, near identical response. In contrast, treatment with a varied set of clinically approved drugs (see Table 2), induces alterations in cell phenotypes that are shown by changes in the fluorescence patterns of one, or more, of the set of cell painting probes (FIGS. 4A-6C). Drugs that have a common mechanism of action, such as DNA damage via topoisomerase inhibition (e.g., etoposide and irinotecan), cause similar changes in multiple cell lines, when features in the cell nucleus are analysed with appropriate painting probes (FIGS. 5A-5B). The data also show multiple examples where different iPS cell lines, each derived from a different human donor, vary in their phenotypic response to treatment with the same drug (FIGS. 4A-7).

Furthermore, the data show that the ability to distinguish differences in the response of different cell lines to the same drug, depends upon the choice of cell paint(s) used and cellular features analysed. The resolution of phenotypic response differences is also shown to vary depending on which data analysis method is used (e.g. comparing UMAP with Heat-MAP and induction plot, FIGS. 4A-5B).

### Summary of Observations:

1. Panels of iPS cell lines can be grown in multiwell plate formats and maintained in a pluripotent state during the time course of a drug response assay.
2. Cell lines analysed by cell painting display consistent, tightly clustered responses to the DMSO control, indicating that the phenotypes measured by cell painting are reproducible and reflect a stable cell state and robustness of the assay.
3. The data show consistent and technically reproducible results, again confirming the assay is robust and reliable.
4. Different cell paints, and combinations of cell paints, have a differential ability to reflect cell phenotypes and drug type-specific responses. Thus, some drugs are revealed to affect the same cell lines differently when analysed with one type of paint, while a different paint does not show a difference with the same cells and drug treatments. We conclude that the choice of cell paints is an important empirical determinant of measuring phenotypic response and differential drug-cell interactions between lines.
5. Different drugs with common effects on cells, exemplified by DNA damaging agents, such as etoposide, result in consistent, reproducible responses, changing the phenotypes of the cell lines, as reported by specific paints, in a similar way. We conclude that the painting assay can report consistent, drug-induced responses across cell lines for specific classes of drugs.
6. We observe cases where a single drug provokes a consistent response across multiple cell lines, but also highlights examples of drugs where individual cell lines vary in the phenotypic response elicited by treatment with the same drug, for the same time. We conclude that the library of cell lines can reproducibly report variation in how different lines are affected by treatment with the same compound, indicating that drug-cell interactions can be variable for different lines, potentially reflecting their differential genotypes and gene expression profiles.
7. We conclude that the cell painting assay can identify specific drugs that show variation in phenotypic response induced between different cell lines, as opposed to drugs that induce consistently similar responses in different cell lines. We note that this can potentially alert researchers to there being a higher chance of a drug in development showing differences in response between patients.
8. We show that a panel of genetically distinct cell lines can be stratified according to measurements arising from how they respond to perturbation in a cell painting assay. We note that further analysis of the stratified cell lines can potentially allow identification of molecular markers, e.g. SNPs or other genotypic markers, and/or mRNA or protein expression markers, that can be used as biomarkers to select patient groups for the downstream design of informed clinical trials.
9. Processing of the raw imaging data collected from the cell painting assays shows that using different analytical strategies can improve the ability to extract phenotypic response data describing how different cell lines respond to drug perturbations.

Once cell painting assays have been performed, additional downstream molecular analyses can be performed to identify mechanisms responsible for variation in drug responses, by comparing directly the groups of cell lines that have been stratified by their response to treatments with specific drugs. We hypothesise that the differential response to drug treatment reflects variations between the cells at the genomic and/or epigenomic level, which leads to variations affecting their respective proteomes, causing differences in drug-cell interactions. This can be determined by performing DNA sequence analyses, combined with mass spectrometry-based assays of protein expression levels and post- translational modifications (PTMs) and by systematic analyses of protein-protein interactions and protein complex formation in the respective cell lines.

The mechanism of differential drug response can also be reflected in comparisons of the transcriptomes of the respective stratified cell lines. However, the levels of stable, expressed protein in either a cell, or tissue, do not always correlate in a linear fashion with the levels of mRNA transcribed from their cognate genes (5, 19). For example, while the full extent of RNA-independent protein regulation is not yet understood in detail, the lack of correlation between mRNA and protein levels has already been seen within the library of HipSci cell lines when mapping Quantitative Trait Loci (QTL) at the respective RNA and protein levels (5). This identified multiple examples where protein level QTLs, including loci linked with human disease susceptibility, were not replicated at the RNA expression level, reflecting situations where protein abundance is controlled by post-transcriptional mechanisms. Furthermore, the data indicated that one such post-transcriptional mechanism includes the formation of multi-protein complexes, where the abundance of one rate-limiting protein subunit can alter the stability of other interacting protein subunits, in trans.

In conclusion, we propose that by combining the high-throughput cell painting analysis of panels of distinct cell lines, with comparative molecular analyses of groups of cell lines stratified by their specific response to drug treatments in the cell painting assay, novel insights can be gained for mechanisms of drug-cell interactions. Furthermore, because human iPSC lines reflect at the molecular level the genetic identity of the donor (5), we propose that this cell-based information, derived in the laboratory, can be used to predict, at least in part, potential clinical variations in drug responses between human patients. This cell line stratification methodology can also identify molecular biomarkers, based upon variations in either DNA sequences (e.g. SNPs), or in expression of mRNAs, proteins, PTMs and/or protein complexes, between the stratified cell lines, that can be used to inform patient selection for the design of clinical trials, leading to improvements in the success rate of drug development pipelines.

### REFERENCES

1. Malik, N. & Rao, M. S. A review of the methods for human iPSC derivation. Methods Mol. Biol. 997, 23-33 (2013).
2. Takahashi, K. & Yamanaka, S. Induction of pluripotent stem cells from mouse embryonic and adult fibroblast cultures by defined factors. Cell 126, 663-676 (2006).
3. Takahashi, K. & Yamanaka, S. Induced pluripotent stem cells in medicine and biology. Development 140, 2457-2461 (2013).
4. Kilpinen, H. et al. Common genetic variation drives molecular heterogeneity in human iPSCs. Nature 546, 370-375 (2017).
5. Mirauta, B. A. et al. Population-scale proteome variation in human induced pluripotent stem cells. Elife 9, (2020).
6. Brenes, A., Bensaddek, D., Hukelmann, J., Afzal, V. & Lamond, A. I. The iPSC proteomic compendium. bioRxiv 469916 (2018) doi:10.1101/469916.
7. Rohban, M. H. et al. Systematic morphological profiling of human gene and allele function via Cell Painting. Elife 6, (2017).
8. Bray, M.-A. et al. Cell Painting, a high-content image-based assay for morphological profiling using multiplexed fluorescent dyes. Nat. Protoc. 11, 1757-1774 (2016).
9. Allan, C. et al. OMERO: flexible, model-driven data management for experimental biology. Nat. Methods 9, 245-253 (2012).
10. McKinney, W. Data Structures for Statistical Computing in Python. in Proceedings of the 9th Python in Science Conference 56-61 (SciPy, 2010). doi:10.25080/Majora-92bf 1922-00a.
11. McInnes, L., Healy, J. & Melville, J. UMAP: Uniform Manifold Approximation and Projection for Dimension Reduction. arXiv [stat.ML] (2018).
12. Liao, Y., Wang, J., Jaehnig, E. J., Shi, Z. & Zhang, B. WebGestalt 2019: gene set analysis toolkit with revamped Uls and APIs. Nucleic Acids Res. 47, W199-W205 (2019).
13. McQuin, C. et al. CellProfiler 3.0: Next-generation image processing for biology. PLoS Biol. 16, e2005970 (2018).
14. Thomas, E. A. et al. Antipsychotic drug treatment alters expression of mRNAs encoding lipid metabolism-related proteins. Mol. Psychiatry 8, 983-93, 950 (2003).
15. Roumeliotis, T. I. et al. Genomic Determinants of Protein Abundance Variation in Colorectal Cancer Cells. Cell Rep. 20, 2201-2214 (2017).
16. Yarden, Y. & Sliwkowski, M. X. Untangling the ErbB signalling network. Nat. Rev. Mol. Cell Biol. 2, 127-137 (2001).
17. Roskoski, R., Jr. The ErbB/HER family of protein-tyrosine kinases and cancer. PharmacoL Res. 79, 34-74 (2014).
18. Modjtahedi, H., Cho, B. C., Michel, M. C. & Solca, F. A comprehensive review of the preclinical efficacy profile of the ErbB family blocker afatinib in cancer. Naunyn. Schmiedebergs. Arch. PharmacoL 387, 505-521 (2014).
19. Ly, T. et aL Proteomic analysis of cell cycle progression in asynchronous cultures, including mitotic subphases, using PRIMMUS. Elife 6, (2017).

1. A method of studying eukaryotic cell responses to a test compound, the method comprising:
providing a library of eukaryotic stem cells, or cell lines, obtained from a donor, or donors, wherein each eukaryotic stem cell, or cell line, within the library, is of the same cell type;
contacting each eukaryotic stem cell, or cell line, within the library, with said test compound in the same manner; and
observing how each eukaryotic stem cell, or cell line, within the library, responds phenotypically to the test compound, in order to identify and group cells, or cell lines, which respond similarly to the test compound.

2. A method of stratifying eukaryotic cells, or cell lines, obtained from a donor, or donors, into one or more groups, wherein each of the eukaryotic cells, or the cell lines, are stem cells of the same cell type, the method comprising;
contacting each eukaryotic stem cell, or cell line, with a test compound in the same manner and observing how each eukaryotic stem cell, or cell line, responds to the same test compound; and
stratifying each eukaryotic stem cell, or cell line, into said one or more groups, based on the observed phenotypic response of each eukaryotic stem cell, or cell line.

3. The method of claim 2, wherein each said group comprises or consists of cells, or cell lines, which respond phenotypically similarly to the same test compound.

4. The method of claim 1, wherein the stem cells, or cell lines, comprise, or consist essentially of, induced pluripotent stem cells (iPSCs).

5. The method of claim 1, wherein the cells, or cell lines, are derived from cells obtained from a donor suffering from a disease, or diseases, associated with known, or candidate, mutations.

6. The method of claim 1, wherein the cells, or cell lines, have been artificially modified in order to alter the genotype.

7. The method of claim 1, wherein observing a response comprises comparing an initial cell, or cell line condition prior to contacting with a test compound, with a cell, or cell line condition subsequent to contacting with a test compound.

8. The method of claim 7, wherein the condition comprises an image-based phenotypic measurement of the cells, or cell lines; preferably a measurement of one or more subcellular components.

9. The method of claim 7, wherein the condition comprises a molecular description of the cells, or cell lines.

10. The method of claim 1, wherein the test compound comprises one or more of a small molecule drug compound, or candidate drug compound, or natural product; a protein/peptide, or RNA macromolecule; an agent for genetically modifying the cell, or cell line, by, for example, gene product depletions, gene mutations, knockouts and knock-ins;
or an infectious agent.

11. The method of claim 1, wherein the method is carried out to profile anticipated differential responses in the human population pertaining to relevant clinical issues, such as drug efficacy, sensitivity, biomarker variation, or sensitivity to infectious agents.

12. The method of claim 1, further comprising further associating the grouped cells with genetic metadata.

13. The method of claim 1, further comprising analysing the grouped cells to identify and characterise protein signatures correlated with defined responses to specific perturbations.

14. The method of claim 1, wherein the method is used in the design of a clinical trial.

15. A method for selecting a patient for participation in a clinical trial, the method comprising performing the method of claim 1 on donor cells, or cell lines, to obtain a grouped cell population; comparing a feature of a potential clinical trial subject with a corresponding feature of the stratified, or grouped cells; and either selecting, or not selecting, the subject for the clinical trial, based on a similarity to that feature of the grouped cells.

16. A method for selecting a therapeutic intervention for a patient, the method comprising performing the method of claim 1, on donor cells, or cell lines, to obtain a grouped cell population; comparing a feature of the patient with a corresponding feature of the grouped cells; and selecting an appropriate therapeutic intervention for the patient, based on a similarity to that feature of the grouped cells.

17. The method of claim 2, wherein the stem cells, or cell lines, comprise, or consist essentially of, induced pluripotent stem cells (iPSCs).

18. The method of claim 2, wherein observing a response comprises comparing an initial cell, or cell line condition prior to contacting with a test compound, with a cell, or cell line condition subsequent to contacting with a test compound.

19. The method of claim 18, wherein the condition comprises an image-based phenotypic measurement of the cells, or cell lines; preferably a measurement of one or more subcellular components.

20. The method of claim 2, further comprising further associating the grouped cells, with genetic metadata.
